(19) United States
(12) Patent Application Publication
(10) Pub. No.: US 2006/0159642 A1
(43) Pub. Date: Jul. 20, 2006

(54) Mascara composition and method of using

(75) Inventors: Fifi G. Hanna, Kearny, NJ (US); Lisa Lamberty, Hawthorne, NJ (US)

Correspondence Address: Charles N.J. Ruggiero, Esq.;Ohlandt, Greeley, Ruggiero & Perle, L.L.P., 10th Floor, One Landmark Square, Stamford, CT, 06901-2682, US

(73) Assignee: Avon Products, Inc.

(21) Appl. No.: 11/019,438

(22) Filed: Dec. 22, 2004

(51) Int. Cl. A61K 8/81

(52) U.S. Cl. 424/70.7

(57) ABSTRACT

There is provided an emulsion mascara composition. The composition has an imide polymer having film forming properties, a wax, water, and a pigment or colorant. The imide polymer has the following structure: 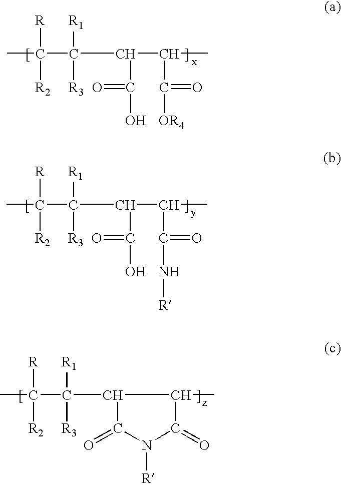 There is also provided a method for imparting curl and adding volume to eyelashes.

### BACKGROUND OF THE INVENTION

1. Field of the Invention

The present invention relates to a mascara composition. The present invention also relates to a method for imparting curl and adding volume to eyelashes.

2. Description of the Related Art

Mascara compositions are commonly employed by women to highlight and enhance the appearance of eyelashes. Such enhancements may include the impartation of color or tone, volume, i.e., thickness and length, and curl.

It would be desirable to have a mascara composition that imparts an enhanced degree of curl and volume to eyelashes for an extended period of time. It would also be desirable to have a method of imparting an enhanced degree of curl and volume to eyelashes for an extended period of time.

### SUMMARY OF THE INVENTION

It is an object of the present invention to provide a mascara composition that imparts an enhanced degree of curl and volume to eyelashes.

It is another object of the present invention to provide a mascara composition that imparts an enhanced degree of curl and volume to eyelashes for an extended period of time.

It is a still another object of the present invention to provide an emulsion mascara composition. The emulsion composition has a film former of an imide polymer, a wax, water, and a pigment or colorant.

It is a further object of the present invention to provide a method for imparting an enhanced degree of curl and volume to eyelashes. The method has the step of applying to the eyelashes an emulsion mascara composition having a film former of an imide polymer, a wax, water, and a pigment or colorant.

### DETAILED DESCRIPTION OF THE INVENTION

It was surprisingly found that there could be a mascara composition that provides an enhanced degree of curl and volume. It was further surprisingly found that the enhanced degree of curl and volume could be provided for an extended period of time. It was also surprisingly found that there could be a method for imparting an enhanced degree of curl and volume for an extended period of time to eyelashes.

An important feature of the mascara composition of the present invention is the presence of an imide polymer. The imide polymer acts as a film former on the exterior surface of the eyelashes. After the composition is applied to the eyelashes, water and other liquid solvents begin to evaporate and cause a retraction/contraction or pulling effect within the film former. The retraction/contraction effect of the film former causes the eyelashes to curl. After the water and any other liquid solvents have evaporated and the formed film has substantially dried, the curl is set.

Useful imide polymers are formed from the following monomeric units:

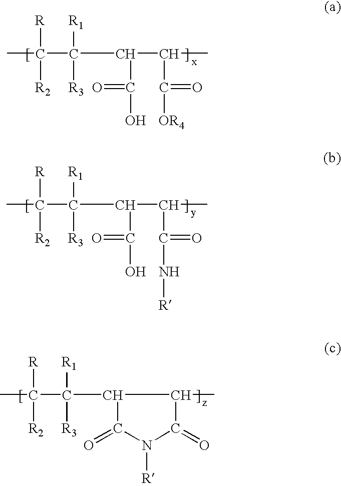

wherein R, R₁, R₂ and R₃ are selected from H, alkyl, alkoxy, cycloalkyl, aryl, easter, acid, fluoro and silyl and R₄ is H or alkyl, preferably alkyl. R′ is hydrogen, aryl, alkyl or alkyl derivatized with fluoro, silyl, amino or olefinic and x=0.05 to 0.95, y=0 to 0.9 and z=0.05 to 0.95. The letters x, y, and z are expressed as mole fractions.

The imide polymers can be formed by reacting, in alcohol solution, R₄OH, a maleic anhydride copolymer or its corresponding half ester with an a-unsubstituted primary amine, R′NH₂.

The reaction is carried out at a temperature of about 60° C. to 160° C., for a predetermined period of time. The reaction temperature is preferably about 80° C. to about 150° C. and most preferably about 100° C. to about 120° C. The reaction time is about 1 to about 25 hours and most preferably about 2 to about 10 hours.

Preferred starting copolymers useful in making the imide polymers include the following: i) an alkyl vinyl ether-maleic anhydride copolymer, such as methyl vinyl ether-maleic anhydride copolymer, or its corresponding half-ester; ii) isobutylene-maleic anhydride copolymer or its corresponding half-ester; iii) ethylene-maleic anhydride copolymer or its corresponding half-ester; and iv) styrene-maleic anhydride copolymer or its corresponding half-ester.

Preferred a-unsubstituted amines useful in making the imide polymers are C₁ to C₄₀ alkyl α-unsubstituted primary amines, such as n-hexylamine, n-octylamine, and 2-ethylhexylamine. Ammonia, silated primary amines, fluorinated primary amines, halogenated primary amines, unsaturated amines, cyano amines and amphoteric amines also may be used.

Imide polymers having no amide component, i.e., all amide having been converted to the cyclic imide form, can be obtained by carrying out the process at 115° C. for about 5 hours or longer. Such an imide polymer has half-ester and cyclic imide repeat units therein.

Polymerized imide monomeric units may constitute a part or the entirety of an imide polymer according to the percentages set forth above. A particularly preferred imide polymer is a isobutylene/dimethylaminopropylmaleimide/ethoxylated maleimide/maleic acid copolymer. A preferred isobutylene/dimethylaminopropylmaleimide/ethoxylated maleimide/maleic acid copolymer is represented by the following structure:

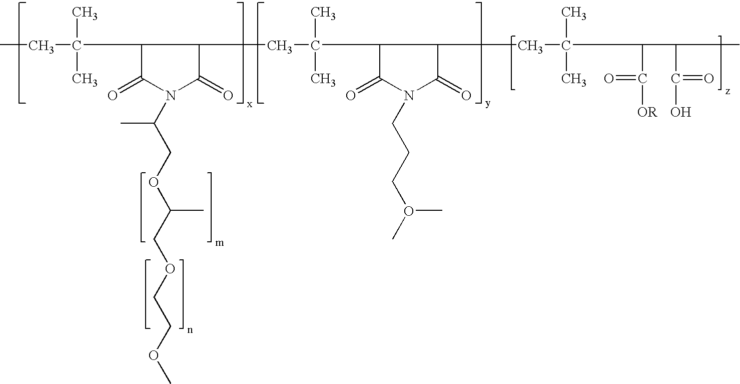

wherein m=1 to 50, n=1 to 50, x=0.05 to 0.95, y=0 to 0.9 and z=0.05 to 0.95 and R═H or CH₂CH₃. The letters x, y, and z are expressed as mole fractions. Isobutylene/dimethylaminopropylmaleimide/ethoxylated maleimide/maleic acid copolymers are preferred film formers. A suitable imide polymer is Aquaflex® XL-30 (sold by ISP)

Additional teachings directed to the structure and composition of useful imide polymers as well as methods for making, are set forth in U.S. Pat. Nos. 5,869,695 and 5,994,385, which are incorporated herein by reference in their entirety. Methods for making the imide polymers are not a critical aspect of the invention.

The imide polymer is present in a film-forming effective amount. Preferably, the imide polymer is present in an amount about 0.05 wt % to about 8 wt % based on the total weight of the composition. More preferably, the imide polymer is present in an amount about 0.1 wt % to about 5 wt % based on the total weight of the composition. Most preferably, the imide polymer is present in an amount about 0.4 wt % to about 1.5 wt % based on the total weight of the composition.

The mascara composition has one or more waxes therein. Suitable waxes include any known in the cosmetic art, such as those of animal origin, plant origin, mineral origin, and synthetic origin. Suitable waxes include, but are not limited to, rice bran wax, carnauba wax, ouricurry wax, candelilla wax, montan wax, sugar cane wax, polyethylene wax, beeswax, microcrystalline wax, or any combination thereof.

Suitable waxes have a melting point in the range from 60° C. to 110° C. and a needle penetration in the range from 1 to 7.5. The needle penetration of waxes is determined according to ASTM D 1321. Mixtures of hard and soft waxes are preferred. A mixture of carnauba wax and beeswax is most preferred.

The wax is present in the mascara composition in an amount effective to enhance the volume of the eyelashes. Preferably, the wax is present at about 8 wt % to about 35 wt % based on the total weight of the composition. More preferably, the wax is present at about 10 wt % to about 30 wt %. Most preferably, the wax is present at about 15 wt % to about 25 wt % based on the total weight of the composition.

The mascara composition has one or more pigments, pearlescents, and/or colorants therein. Useful pigments and/or colorants include any known in the cosmetic art. Coatings and surface treatments may also enhance the shine or gloss exhibited by mascara compositions. Examples of useful pigments include titanium dioxide, zinc oxide, iron oxide, chromium oxide, ferric blue, and mica; organic pigments include barium, strontium, calcium or aluminium lakes, ultramarines, and carbon black; colorants include D&C Green #3, D&C Yellow #5, and D&C Blue #1. Pigments and/or colorants may be coated or surface treated with one or more compatibilizers to aid in dispersion in either or both of the aqueous or wax phases. Preferred pigments and/or colorants are those surface treated with dimethicone copolyol.

Pigments can be present in the composition in an amount of 1 to about 25 wt. % of the total weight of the composition, and preferably in an amount of about 1 to about 15 wt. %.

The term “pearlescent agents” should be understood as meaning iridescent particles, in particular, particles produced by certain molluscs in their shell or synthesized pearlescent particles. These pearlescent agents particularly serve to modify the texture of the composition.

Pearlescent agents can be present in the composition in an amount from 0 to about 20 wt % based on the total weight of the composition and preferably an amount from about 1 wt % to about 15 wt %. Suitable pearlescent agents also include mica coated with titanium oxide, with iron oxide, or with a natural pigment.

The mascara composition optionally may have film formers in addition to the imide polymer (“additional film formers”). An additional film former enables desirable properties, such as degree and longevity of curl of the eyelashes, to be enhanced and/or optimized. One or more additional film formers may be substantially water-resistant or oil-resistant and be of natural or synthetic origin. Suitable oil-resistant film formers include, but not limited to the following, acrylics (acrylates), polyacrylates, acrylamide polymers and copolymers and quaternary salts thereof, urethanes, polyurethanes, polyesters, polysaccharides, polyamides, polyols, polyethers, cellulosics, proteins, polyamino acids, esters derived from rosin, latexes, or any combinations thereof. Suitable water-resistant film formers include, but not limited to, polyolefins, polyvinylpyrrolidone polymers and copolymers, polyethylenes, polyalkyls, polystyrenes, triglycerides, epoxy resins, shellacs, or any combinations thereof.

A preferred composition has an additional film former that is substantially water-resistant. A preferred water-resistant film former is a vinylpyrrolidone/eicosene copolymer. A suitable vinylpyrrolidone/eicosene copolymer is Ganex® V-220 (marketed by ISP).

The water-resistant film former, if present, is present preferably at about 0.1 wt % to about 1.5 wt % based on the total weight of the composition. More preferably, the water-resistant film former is present at about 0.1 wt % to about 1 wt %. Most preferably, the water-resistant film former is present at about 0.2 wt % to about 0.6 wt %.

A preferred composition also has an additional film former that is a cationic polymer. Suitable cationic polymers include, but are not limited to, Polyquaternium-4, Polyquaternium-5, Polyquaternium-6, Polyquaternium-7, Polyquaternium-10, Polyquaternium-22, Polyquaternium-37, Polyquaternium-47, or any combination thereof. Polyquaternium-7 is especially preferred. Polyquaternium-7 is a quaternary ammonium salt of a acrylamide/dimethyl diallyl ammonium chloride copolymer. Polyquaternium-7 is available as SALCARE® Super 7 (marketed by Ciba Specialty Chemicals, Inc.).

The cationic polymer film former, if present, is present preferably at about 0.1 wt % to about 2 wt % based on the total weight of the composition. More preferably, the cationic polymer film former is present at about 0.1 wt % to about 1.5 wt %. Most preferably, the cationic polymer film former is present at about 0.25 wt % to about 1 wt %.

Preferred compositions have an imide polymer film former, a water-resistant film former, and cationic polymer film former. Most preferred compositions have film formers of an isobutylene/dimethylaminopropylmaleimide/ethoxylated maleimide/maleic acid copolymer, a vinylpyrrolidone/eicosene copolymer, and a quaternary ammonium salt of an acrylamide/dimethyl diallyl ammonium chloride copolymer.

In preferred compositions, the ratio of imide polymer film former to water-resistant film former is about 15:1 to about 1:1 and more preferably about 6:1 to about 2:1. The ratio of imide polymer film former to cationic polymer film former is about 20:1 to about 1:1 and more preferably about 15:1 to about 5:1.

The mascara composition of the present invention optionally has a multiplicity of fibers therein. When the mascara composition is applied, the fibers are laid on the surfaces of the eyelashes. The fibers enhance the volume of the eyelashes, i.e., increase thickness and length thereof. The fibers may be of any type known in the cosmetic art and may be natural or synthetic. Thermoplastic fibers, such as those of polyethylene, polypropylene, or nylon, are preferred.

Fibers are present in the mascara composition in an amount effective to enhance volumizing capability. Preferably, fibers are present in an amount about 0.05 wt % to about 1 wt % based on the total weight of the composition. More preferably, the fibers are present at about 0.1 wt % to about 0.7 wt %. Most preferably, the fibers are present at about 0.2 wt % to about 0.5 wt %.

The mascara composition of the present invention takes the form of an emulsion. The emulsion may be a wax-in-water or water-in-wax emulsion. A wax-in-water emulsion is preferred. The wax phase may alternately be characterized as an oil phase. The composition has one or more emulsifiers/surfactants. Preferred emulsifiers are prepared in-situ via reaction between a base, preferably triethanolamine, and an organic acid, preferably stearic acid. The emulsifier is present in an amount sufficient to maintain a stable emulsion. Preferably, the emulsifier is present at about 1 wt % to about 20 wt % and most preferably at about 2.5 wt % to about 10 wt % based on the total weight of the composition. The composition is aqueous and preferably has 30 wt % to about 75 wt %, more preferably 40 wt % to about 65 wt %, and most preferably 45 wt % to about 60 wt %, water based on the total weight of the composition.

The mascara composition may optionally have one or more additional vehicles (in addition to water) that are cosmetically acceptable. Such additional vehicles include, but are not limited to, one or more lower alcohols, fatty alcohols, fatty ethers, fatty esters, isododecane, polyols, glycols, liposomes, laminar lipid materials, or any combinations thereof.

The aqueous phase is a mixture of one or more water soluble or water dispersible ingredient, which can be liquid, semi-solid or solid at room temperature (25° C.). A person skilled in the art can select the appropriate cosmetic form, the ingredients contained therein, as well as the method for preparing it, on the basis of the knowledge that the skilled artisan possesses.

The composition of the invention may further have any ingredient conventionally used in the cosmetic field, in particular in the manufacture of mascara products. The amounts of these various ingredients are those conventionally used in the cosmetic field to achieve their intended purpose, and range typically from about 0.01 to about 20 wt. % by weight of the composition. The nature of these ingredients and their amounts must be compatible with the production of stable compositions of the invention.

The composition of the invention may also comprise fillers conventionally used in cosmetic compositions. The term “fillers” should be understood as meaning colorless or white, inorganic or synthetic, lamellar or non-lamellar particles.

Fillers may be present in an amount from 0 to about 30 wt. % by weight of the composition, preferably about 0.1 to about 15 wt. %. Suitable fillers include talc, silica, zinc stearate, mica, kaolin, nylon powder, polyethylene powder, Teflon, starch, boron nitride, copolymer microspheres such as Expancel (Nobel Industrie), Polytrap (Dow Coming), and silicone resin microbeads (Tospearl from Toshiba).

Compositions may also include one or more of the following optional ingredients: amino acids, antioxidants, emollients, emulsifiers, eyelash protectants, eyelashes penetration enhancers, humectants, moisturizers, preservatives, surfactants, thickeners, viscosity and/or rheology modifiers, vitamins, or any combinations thereof.

The mascara composition may be applied to the eyelashes as often as needed or desired to impart curl, volume, and color. Typically, the composition will be applied once per day. If desired the composition may be applied twice or more per day. The composition will provide curl retention and longevity as well as volumizing for preferably about 8 or more hours per day, more preferably about 10 or more hours per day, and most preferably about 12 or more hours per day. Longevity means exhibiting substantially no flaking or brittleness on the lash and exhibiting substantially no smudging or smearing.

The following are examples of the present invention. Unless otherwise indicated, all percentages or parts are by weight.

### EXAMPLES

Mascara compositions of the present invention were prepared. Ingredients are set forth in Table 1.

TABLE 1

| | 1* | 2* | 3* |
|---|---|---|---|
| Ingredients | (Wt %) | (Wt %) | (Wt %) |
| Triethanolamine stearate | 7.0 | 6.5 | 8.0 |
| Beeswax | 14.0 | 15 | 25 |
| Carnauba wax | 9 | 5.0 | 2.5 |
| Candelilla wax | 0 | 2.5 | 2.5 |
| Gum Arabic | 1.0 | 1.4 | 0.8 |
| Methylparaben | 0.4 | 0.45 | 0.5 |
| Propylparaben | 0.2 | 0.25 | 0.3 |
| Phenoxyethanal | 0.2 | 0.3 | 0.35 |
| Polyimide Polymer¹ | 0.1 | 1.5 | 8.0 |
| VP/Eicosene Copolymer² | 0.1 | 1.0 | 1.5 |
| Polyquaternium-7³ | 0.1 | 1.0 | 1.5 |
| Black iron oxide | 8.0 | 9.0 | 7.0 |
| Ultramarines | 1.0 | 0.20 | 1.0 |
| Polypropylene fiber | 0.1 | 0.4 | 1.5 |
| Water (Demineralized) | QS 100% | QS 100% | QS 100% |

*Percents are provided on an active ingredient basis
¹Aquaflex XL-30 (ISP - 30% active)
²Ganex -220 (ISP - 100% active)
³Salcare Super 7 (Ciba Geigy Specialty Chemicals - 40% active)

The wax phase is prepared by melting the lipophilic ingredients (the waxes, triethanolamine stearate, and the VP/Eicosene copolymer) at 85° C. to 110° C. to form an oil premix. The hydrophilic ingredients (polyimide polymer, Polyquaterium-7, gum arabic and preservatives) and the pigments are admixed with water at 85° C. to 110° C. to form a water phase premix. The premixes are combined usually by adding the oil phase to the water phase with high speed mixing, and the resulting mixture cooled to room temperature. A conventional mascara container is then filled with the composition and the container is sealed with cap including a mascara applicator brush.

It should be understood that the foregoing description is only illustrative of the present invention. Various alternatives and modifications can be devised by those skilled in the art without departing from scope of the invention. Accordingly, the present invention is intended to embrace all such alternatives, modifications and variances that fall within the scope of the appended claims.

What is claimed is

1. A mascara composition, comprising:
an imide polymer having film forming properties;
a wax;
water; and
a pigment, pearlescent or colorant,
wherein the composition is an emulsion; and wherein the imide polymer has the following structure: 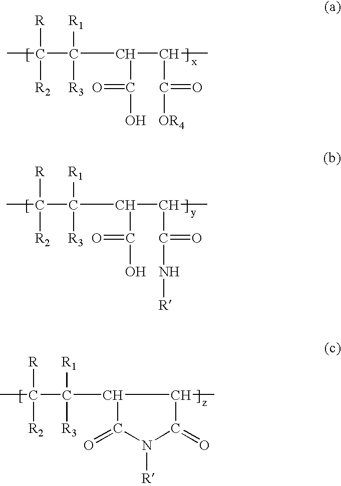
wherein R, R₁, R₂ and R₃ are selected from H, alkyl, alkoxy, cycloalkyl, aryl, ester, acid, fluoro and silyl and R₄ is alkyl; wherein R′ is hydrogen, aryl, alkyl or alkyl derivatized with fluoro, silyl, amino or olefinic and x=0.05 to 0.95, y=0 to 0.9 and z=0.05 to 0.95.

2. The composition of claim 1, wherein the imide polymer is present in an amount about 0.05 to about 8 based on the total weight of the composition.

3. The composition of claim 1, wherein the imide polymer is present in an amount about 0.4 to about 1.5 based on the total weight of the composition.

4. The composition of claim 1, wherein the wax is present in an amount effective to enhance the volume of eyelashes.

5. The composition of claim 1, wherein the wax is present about 8 wt % to about 35 wt % based on the total weight of the composition.

6. The composition of claim 1, wherein the wax is present about 15 wt % to about 25 wt % based on the total weight of the composition.

7. The composition of claim 1, wherein the wax is one or more waxes selected from the group consisting of rice bran wax, carnauba wax, ouricurry wax, candelilla wax, montan waxes, sugar cane waxes, polyethylene waxes, beeswax, microcrystaline wax, and any combination thereof.

8. The composition of claim 1, wherein the wax is a mixture of hard and soft waxes.

9. The composition of claim 8, wherein the mixture has carnauba wax and beeswax.

10. The composition of claim 1, wherein the emulsion is a wax-in-water emulsion.

11. The composition of claim 1, wherein the water is present at about 30 wt % to about 75 wt % based on the total weight of the composition.

12. The composition of claim 1, wherein the water is present at about 45 wt % to about 60 wt % based on the total weight of the composition.

13. The composition of claim 1, further comprising an additional film former that is substantially water-resistant.

14. The composition of claim 13, wherein the substantially water-resistant film former is present in an amount about 0.1 wt % to about 1.5 wt % based on the total weight of the composition.

15. The composition of claim 13, wherein the substantially water-resistant film former is present in an amount about 0.2 wt % to about 0.6 wt % based on the total weight of the composition.

16. The composition of claim 13, wherein the substantially water-resistant film former is selected from the group consisting of polyolefins, polyvinylpyrrolidone polymers and copolymers, polyethylenes, polyalkyls, polystyrenes, triglycerides, epoxy resins, shellacs, and any combinations thereof.

17. The composition of claim 13, wherein the substantially water-resistant film former is a vinylpyrrolidone/eicosene copolymer.

18. The composition of claim 1, further comprising an additional film former that is a cationic polymer.

19. The composition of claim 18, wherein the cationic polymer film former is present in an amount about 0.1 wt % to about 2 wt % based on the total weight of the composition.

20. The composition of claim 18, wherein the cationic polymer film former is present in an amount about 0.25 wt % to about 1 wt % based on the total weight of the composition.

21. The composition of claim 18, wherein the cationic polymer film former is selected from the group consisting of Polyquaternium-4, Polyquaternium-5, Polyquaternium-6, Polyquaternium-7, Polyquaternium-10, Polyquaternium-22, Polyquaternium-37, Polyquaternium-47, and any combination thereof.

22. The composition of claim 1, further comprising a water-resistant film former and a cationic polymer film former.

23. The composition of claim 1, further comprising a multiplicity of fibers, the multiplicity fibers being present at from about 0.05 to about 1 wt % based on the weight of the composition.

24. The composition of claim 1, wherein the pigment, pearlescent or colorant is a pigment present in an amount about 1 to about 30 wt % based on the weight of the composition.

25. The composition of claim 1, wherein the imide polymer is present in an amount about 0.05 wt % to about 8 wt % based on the total weight of the composition, and wherein the wax is present in an amount about 8 wt % to about 35 wt % based on the total weight of the composition, and wherein the emulsion is a wax-in-water emulsion, and wherein the water is present at about 30 wt % to about 75 wt % based on the total weight of the composition, and wherein the composition further comprises a multiplicity of fibers, a substantially water-resistant film former at about 0.1 wt % to about 1.5 wt % based on the total weight of the composition, a cationic polymer film former at about 0.1 wt % to about 2 wt % based on the total weight of the composition, and a multiplicity of fibers present at about 0.5 to about 1 wt % based on the weight of the composition.

26. The composition of claim 25, wherein the imide polymer is an isobutylene/dimethylaminopropylmaleimide/ethoxylated maleimide/maleic acid copolymer, wherein the substantially water-resistant film former is a vinylpyrrolidone/eicosene polymer, and wherein the cationic polymer film former is a quaternary ammonium salt of a acrylamide/dimethyl diallyl ammonium chloride copolymer.

27. A mascara composition, comprising:
an imide polymer having film forming properties;
a wax;
water; and
a pigment or colorant,
wherein the composition is an emulsion; and wherein the imide polymer has the following structure: 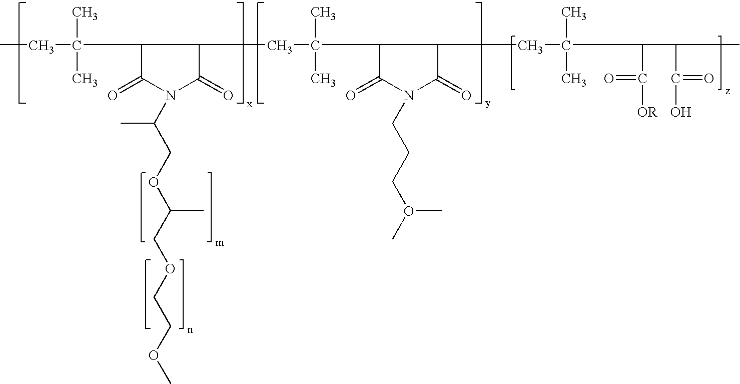
wherein m=1 to 50, n=1 to 50, x=0.05 to 0.95, y=0 to 0.9 and z=0.05 to 0.95 and R═H or CH₂CH₃.

28. A method for imparting curl and adding volume to eyelashes, comprising topically applying to the eyelashes a mascara composition having an imide polymer having film forming properties, a wax, water, and a pigment or colorant, wherein the composition is an emulsion, and wherein the imide polymer has the following structure: 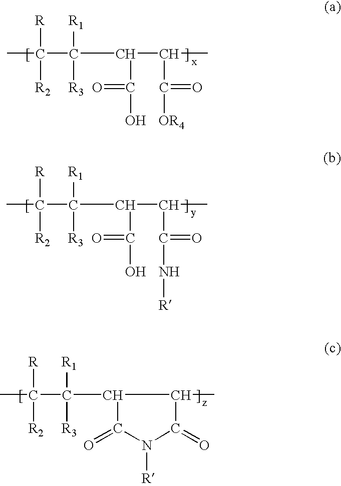
wherein R, R₁, R₂ and R₃ are selected from H, alkyl, alkoxy, cycloalkyl, aryl, ester, acid, fluoro and silyl and R₄ is alkyl; wherein R′ is hydrogen, aryl, alkyl or alkyl derivatized with fluoro, silyl, amino or olefinic and x is 0.05-0.95, y=0-0.9 and z=0.05-0.95.

29. The method of claim 28, wherein the curl and volume are retained for about 8 or more hours.

30. A method for imparting curl and adding volume to eyelashes, comprising topically applying to the eyelashes a mascara composition having an imide polymer having film forming properties, a wax, water, and a pigment or colorant, wherein the composition is an emulsion, and wherein the imide polymer has the following structure: 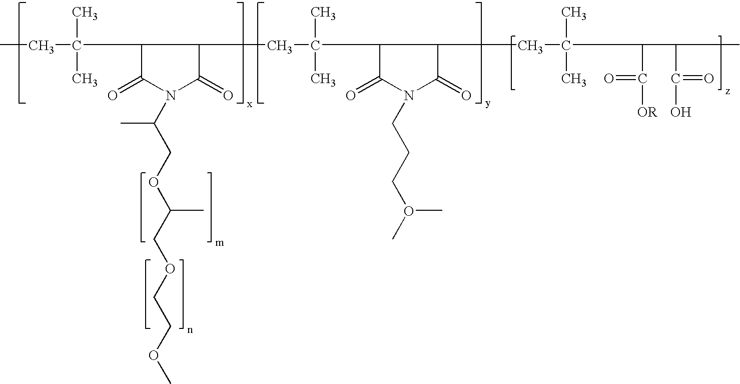
wherein m1 to 50, n=1 to 50, x=0.05 to 0.95, y=0 to 0.9 and z=0.05 to 0.95 and R═H or CH₂CH₃.

31. A mascara composition, comprising:
an imide polymer having film forming properties;
a wax;
water; and
a pigment, pearlescent or colorant,
wherein the composition is an emulsion.

32. The composition of claim 25, wherein the imide polymer is an isobutylene/dimethylaminopropylmaleimide/ethoxylated maleimide/maleic acid copolymer.
